(19) United States
(12) Patent Application Publication
(10) Pub. No.: US 2003/0013768 A1
(43) Pub. Date: Jan. 16, 2003

(54) Process for the production of sertraline and intermediates useful therefor

(75) Inventors: Ilpo Laitinen, Espoo (FI); Pekka Pietikaeinen, Espoo (FI)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: ORION COPRORATION FERMION, Espoo (FI)

(21) Appl. No.: 10/158,114

(22) Filed: May 31, 2002

Related U.S. Application Data
(60) Provisional application No. 60/294,266, filed on May 31, 2001.

(51) Int. Cl. C07C 249/02; A61K 31/13

(52) U.S. Cl. 514/641; 564/270

(57) ABSTRACT

A pharmaceutical intermediate, N-[4-(3,4-dichlorophenyl)-3,4-dihydro-1(2H)-naphthalenylidene]methanamine, which can be used in the production of sertraline hydrochloride, is conveniently prepared by reacting 4-(3,4-dichlorophenyl)-3,4-dihydro-1-(2H)-naphthalenone with monomethylamine in a solvent which is an amide solvent with a structure of general formula IV:

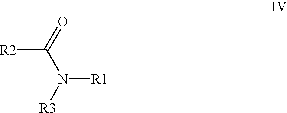

wherein R1, R3 are independently hydrogen or C₁₋₆ alkyl, which can be substituted, and R2 is hydrogen.

### BACKGROUND OF THE INVENTION

1. Field of the Invention

The present invention relates to a novel method for the production of sertraline. The present invention also relates to a novel process for the preparation of a pharmaceutical intermediate, N-[4-(3,4-dichlorophenyl)-3,4-dihydro-1 (2H)-naphthalenylidene]methanamine, which is useful for the production of sertraline.

2. Discussion of the Background

N-[4-(3,4-dichlorophenyl)-3,4-dihydro-1 (2H)-naphthalenylidene]-methanamine of formula I:

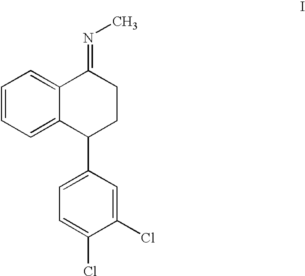

is a well known pharmaceutical intermediate which can be used, e.g., in the preparation of sertraline, (1S-cis)-4-(3,4-dichlorophenyl)-1,2,3,4-tetrahydro-N-methyl-1-naphthalenamine, which has the structure of formula II:

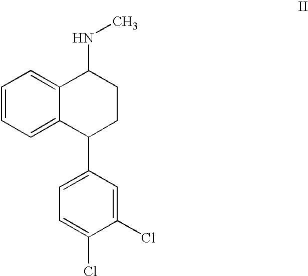

Sertraline is marketed in the form of its hydrochloride for the treatment of depression, obsessive-compulsive disorder and panic disorder.

The synthesis of sertraline is described in U.S. Pat. No. 4,536,518. The process described includes a condensation reaction of 4-(3,4-dichlorophenyl)-3,4-dihydro-1(2H)-naphthalenone of formula III:

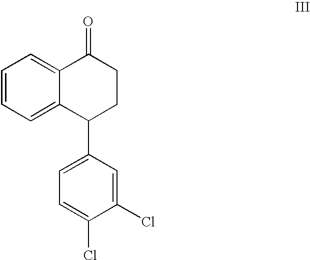

with monomethylamine, which is catalyzed by titanium tetrachloride yielding N-[4-(3,4-dichlorophenyl)-3,4-dihydro-1(2H)-naphthalenylidene]methanamine. The reaction is an equilibrium reaction, in which the equilibrium has to be shifted. This can be done, e.g., by using titanium tetrachloride to remove water from the reaction mixture. Titanium tetrachloride, however, is extremely reactive with water, and the side products formed are hazardous, and therefore other dehydrating agents have been considered.

Another route to N-4-[3,4-dichlorophenyl)-3,4-dihydro-1 (2H)-naphthalenylidene]methanamine is described in U.S. Pat. No. 4,855,500, in which the dehydration characteristics of appropriate mesh molecular sieves are employed to remove water from the reaction mixture to promote the condensation reaction between 4-(3,4-dichlorophenyl)-3,4-dihydro-1(2H)-naphthalenone and monomethylamine. However, molecular sieves are expensive and they must typically be regenerated if they are to be reused.

In a process described in EP 1 059 287, the (+) enantiomer of sertraline is prepared by either of the processes described above using the (+) enantiomer of 4-(3,4-dichlorophenyl)-3,4-dihydro-1(2H)-naphthalenone as a starting material, so that no optical resolution of the final product is required.

Still another route to to N-4-[3,4-dichlorophenyl)-3,4-dihydro-1 (2H)-naphthalenylidene]methanamine is described in the published PCT patent application WO 99/36394. In the process described, the condensation reaction of 4-(3,4-dichlorophenyl)-3,4-dihydro-1(2H)-naphthalenone with monomethylamine is performed in an alcohol solvent. The solubility of the reaction product in the reaction solvent is such that the equilibrium is favorably enhanced towards the product. No catalysts or dehydrating agents are required. However, monomethylamine is easily vaporized at the reaction temperatures (about 50° C. or above), and therefore the reaction is carried out under pressure and a suitable pressure rated vessel is needed.

Sertraline hydrochloride is produced by further hydrogenating the N-4-[3,4-dichlorophenyl)-3,4-dihydro-1(2H)-naphthalenylidene]methanamine resulting from processes described above and resolving the racemic mixture and finally crystallizing sertraline hydrochloride.

Thus, there remains a need for improved processes for preparing sertraline and sertraline hydrochloride. There also remains a need for improved processes for preparing, N-[4-(3,4-dichlorophenyl)-3,4-dihydro-1(2H)-naphthalenylidene]-methanamine.

### SUMMARY OF THE INVENTION

Accordingly, it is one object of the present invention to provide novel processes for preparing sertraline and sertraline hydrochloride.

It is another object of the present invention to provide novel processes for preparing sertraline and sertraline hydrochloride which afford sertraline and sertraline hydrochloride in good yield.

It is another object of the present invention to provide novel processes for preparing sertraline and sertraline hydrochloride which are convenient to carry out.

It is another object of the present invention to provide novel processes for preparing sertraline and sertraline hydrochloride which do not employ expensive and/or hazardous reagents.

It is another object of the present invention to provide novel processes for preparing sertraline and sertraline hydrochloride which produce impurities in reduced amounts.

It is another object of the present invention to provide novel processes for preparing sertraline and sertraline hydrochloride which may be carried out under atmospheric pressure and at ambient temperatures.

It is another object of the present invention to provide novel processes for preparing N-[4-(3,4-dichlorophenyl)-3,4-dihydro-1(2H)-naphthalenylidene]-methanamine.

It is another object of the present invention to provide novel processes for preparing N-[4-(3,4-dichlorophenyl)-3,4-dihydro-1(2H)-naphthalenylidene]-methanamine which afford N-[4-(3,4-dichlorophenyl)-3,4-dihydro-1(2H)-naphthalenylidene]methanamine in good yield.

It is another object of the present invention to provide novel processes for preparing N-[4-(3,4-dichlorophenyl)-3,4-dihydro-1(2H)-naphthalenylidene]-methanamine which are convenient to carry out.

It is another object of the present invention to provide novel processes for preparing N-[4-(3,4-dichlorophenyl)-3,4-dihydro-1(2H)-naphthalenylidene]-methanamine which do not employ expensive and/or hazardous reagents.

It is another object of the present invention to provide novel processes for preparing N-[4-(3,4-dichlorophenyl)-3,4-dihydro-1(2H)-naphthalenylidene]-methanamine which produce impurities in reduced amounts.

It is another object of the present invention to provide novel processes for preparing N-[4-(3,4-dichlorophenyl)-3,4-dihydro-1(2H)-naphthalenylidene]-methanamine which may be carried out under atmospheric pressure and ambient temperature.

These and other objects, which will become apparent during the following detailed description, have been achieved by the inventor's surprising discovery that if the solvent for the imination of 4-(3,4-dichlorophenyl)-3,4-dihydro-1-(2H)-naphthalenone is selected from the solvents of the invention described below the reaction can be performed under atmospheric pressure and at ambient temperature. In addition, the amount of the solvent used is reduced, impurities are not formed, and the yield is good. Moreover, water removal agents like titanium tetrachloride or molecular sieves are not required.

Thus, in a first embodiment, the present invention provides a process for preparing N-[4-(3,4-dichlorophenyl)-3,4-dihydro-1(2H)-naphthalenylidene]methanamine, said process comprising:

(1) reacting 4-(3,4-dichlorophenyl)-3,4-dihydro-1-(2H)-naphthalenone of formula III:

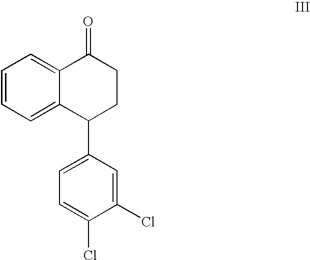

with monomethylamine in a solvent which is selected from the group consisting of amide solvents with a structure of general formula IV:

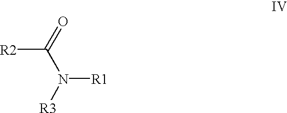

wherein R1 and R3 are independently hydrogen or C₁₋₆ alkyl, which can be substituted and R2 is hydrogen.

In another embodiment, the present invention provides a process wherein the N-[4-(3,4-dichlorophenyl)-3,4-dihydro-1(2H)-naphthalenylidene]methanamine so formed in the process of the invention is hydrogenated to form sertraline which may be further resolved by the use of, e.g., mandelic acid and finally crystallized as (1S-cis)-4-(3,4-dichlorophenyl)-1,2,3,4-tetrahydro-N-methyl-1-naphthalenamine hydrochloride or some other pharmaceutically suitable salt.

In still another embodiment, the present invention provides a pharmaceutical composition comprising (1S-cis)-4-(3,4-dichlorophenyl)-1,2,3,4-tetrahydro-N-methyl-1-naphthalenamine or its hydrochloride or some other pharmaceutically suitable salt prepared by the process of the invention.

### DETAILED DESCRIPTION OF THE PREFERRED EMBODIMENTS

In a first embodiment, the present invention provides a process for producing N-[4-(3,4-dichlorophenyl)-3,4-dihydro-1(2H)-naphthalenylidene]methanamine, by reacting 4-(3,4-dichlorophenyl)-3,4-dihydro-1-(2H)-naphthalenone with monomethylamine in a solvent selected from the a group consisting of amide solvents of general formula IV:

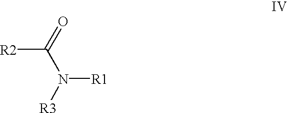

wherein R1, and R3 are independently hydrogen or C₁₋₆ alkyl, which can be substituted, and R2 is hydrogen.

In a preferred embodiment of the present invention the solvent used is dimethylformamide or methylformamide, most preferably, the solvent is dimethylformamide.

The imination of 4-(3,4-dichlorophenyl)-3,4-dihydro-1-(2H)-naphthalenone with monomethylamine may be performed in the presence of acid catalyst, which can be any suitable organic or inorganic acid, e.g., formic acid, acetic acid, sulfonic acid, or hydrochloric acid. In a preferred embodiment of the invention, formic acid or acetic acid is used as the acid catalyst.

The solubility of the product in the solvent of the invention is low, so that the product is slowly crystallizing out of the reaction mixture and it can be isolated easily by, e.g., filtration. The process has also considerable purification capacity. The reaction can be performed under atmospheric pressure and is typically carried out at a temperature in the range of from about 0° C. to about 50° C., preferably at ambient temperature, i.e., from about 15° C. to about 25° C. Of course, the imination may also be carried out under a slight positive pressure of an inert atmosphere, such as nitrogen gas or argon gas.

Suitably, the 4-(3,4-dichlorophenyl)-3,4-dihydro-1-(2H)-naphthalenone is added to the solvent in an amount of about 300 g to about 400 g per liter of solvent, preferably about 320 g to about 350 g per liter of solvent. The methylamine is added in an amount of about 4 mole to about 6 mole per mole of 4-(3,4-dichlorophenyl)-3,4-dihydro-1-(2H)-naphthalenone, preferably about 4.8 mole to about 5.2 mole per mole of 4-(3,4-dichlorophenyl)-3,4-dihydro-1-(2H)-naphthalenone. When used, the acid catalyst is typically added to the mixture in an amount of about 0.1 mole to about 2.0 mole per mole of 4-(3,4-dichlorophenyl)-3,4-dihydro-1-(2H)-naphthalenone, preferably about 0.4 mole to about 0.6 mole per mole of 4-(3,4-dichlorophenyl)-3,4-dihydro-1-(2H)-naphthalenone.

The present method is not constrained to any particular order of addition, and the reaction may be conveniently performed by charging all of the components into a suitable-size vessel at 0° C. and then allowing the reaction mixture to rise to ambient temperature. The reaction mixture is then stired at ambient temperature for a time of about 10 to about 30 hours, preferably about 20 to about 24 hours. If desired, the progress of the reaction may be monitored by any suitable technique, including chromatography, especially high-pressure liquid chromatography (HPLC) or thin-layer chromatography (TLC).

The resulting imine compound, N-[4-(3,4-dichlorophenyl)-3,4-dihydro-1(2H)-naphthalenylidene]methanamine is insoluble in the reaction solvent and exists as a solid precipitate in the reaction mixture at the completion of the reaction. The resulting imine compound, N-[4-(3,4-dichlorophenyl)-3,4-dihydro-1(2H)-naphthalenylidene]methanamine may then be isolated from the reaction mixture by any suitable solid-liquid separation technique, such as filtration, centrifugation, or decantation.

The resulting imine compound, N-[4-(3,4-dichlorophenyl)-3,4-dihydro-1(2H)-naphthalenylidene]methanamine may be further hydrogenated to form cis-(1S)(1R)-4-(3,4-dichlorophenyl)-1,2,3,4-tetrahydro-N-methyl-1-naphthalenamine which may then be optically resolved with, e.g., mandelic acid and finally crystallized to afford (1S-cis)-4-(3,4-dichlorophenyl)-1,2,3,4-tetrahydro-N-methyl-1-naphthalenamine hydrochloride or some other pharmaceutically suitable salt.

Pharmaceutical compositions comprising (1S-cis)-4-(3,4-dichlorophenyl)-1,2,3,4-tetrahydro-N-methyl-1-naphthalenamine or its pharmaceutically suitable salt prepared by the method of the invention can be prepared by methods well-known in the art.

Other features of the invention will become apparent in the course of the following descriptions of exemplary embodiments which are given for illustration of the invention and are not intended to be limiting thereof.

### EXAMPLES

### Example 1

Preparation of N-[4-(3,4-dichlorophenyl)-3,4-dihydro-1-(2H)-naphthalenylidene]-methanamine.

4-(3,4-Dichlorophenyl)-3,4-dihydro-1-(2H)-naphthalenone (100 g), N,N-dimethylformamide (300 ml), and formic acid (6.5 ml) are charged into a reaction vessel. Methylamine (56.0 g) is then added at about 0° C. The mixture is then stirred for 20 hours at room temperature. The mixture is cooled to 10° C. and stirred for 1 hour. The crystalline compound is collected by filtration and washed with methanol. The yield is 97.7 g (93.5%) as dried.

The same process was performed using N-methylformamide as a solvent in the imination step. Yield was 90.1%.

### Example 2

Preparation of (1S-cis)-4-(3,4-dichlorophenyl)-1,2,3,4-tetrahydro-N-methyl-1-naphthalenamine Hydrochloride (Sertraline Hydrochloride)

N-[4-(3,4-dichlorophenyl)-3,4-dihydro-1(2H)-naphthalenylidene]-methanamine (50 g) is hydrogenated over palladium on charcoal to yield cis-(1S)(1R)-4-(3,4-dichlorophenyl)-1,2,3,4-tetrahydro-N-methyl-1-naphthalenamine. The racemic compound is resolved with mandelic acid and finally crystallized as sertraline hydrochloride. The total yield from 4-(3,4-dichlorophenyl)-3,4-dihydro-1-(2H)-naphthalenone is 67% (of the theoretical (+)-enantiomer).

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A process for preparing N-[4-(3,4-dichlorophenyl)-3,4-dihydro-1(2H)-naphthalenylidene]methanamine of formula I: 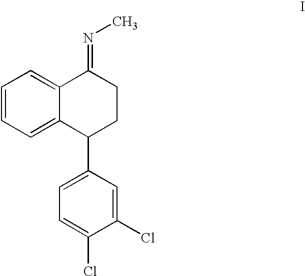
said process comprising:
(1) reacting 4-(3,4-dichlorophenyl)-3,4-dihydro-1-(2H)-naphthalenone of formula III: 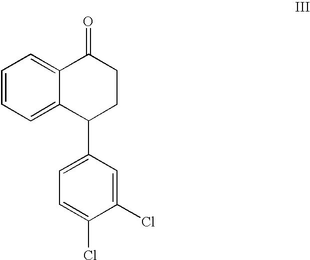
with monomethylamine in a solvent which is selected from the group consisting of amide solvents with a structure of general formula IV: 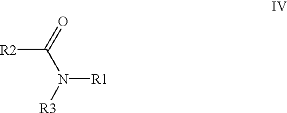
wherein R1 and R3 are independently hydrogen or C₁₋₆ alkyl, which can be substituted and R2 is hydrogen.

2. The process of claim 1, wherein said solvent is selected from the group consisting of dimethylformamide and methylformamide.

3. The process of claim 1, wherein said solvent is dimethylformamide.

4. The process of claim 1, wherein said reaction is performed in the presence of an acid catalyst.

5. The process of claim 4, wherein said acid catalyst is selected from the group consisting of formic acid and acetic acid.

6. The process of claim 2, wherein said reaction is performed in the presence of an acid catalyst.

7. The process of claim 6, wherein said acid catalyst is selected from the group consisting of formic acid and acetic acid.

8. The process of claim 3, wherein said reaction is performed in the presence of an acid catalyst.

9. The process of claim 8, wherein said acid catalyst is selected from the group consisting of formic acid and acetic acid.

10. The process of claim 1, wherein said reaction is carried out at a temperature of from about 0° C. to about 50° C.

11. The process of claim 2, wherein said reaction is carried out at a temperature of from about 0° C. to about 50° C.

12. The process of claim 3, wherein said reaction is carried out at a temperature of from about 0° C. to about 50° C.

13. The process of claim 4, wherein said reaction is carried out at a temperature of from about 0° C. to about 50° C.

14. The process of claim 5, wherein said reaction is carried out at a temperature of from about 0° C. to about 50° C.

15. A process for producing (1S-cis)-4-(3,4-dichlorophenyl)-1,2,3,4-tetrahydro-N-methyl-1-naphthalenamine or pharmaceutically acceptable salt thereof, which has the structure of formula II: 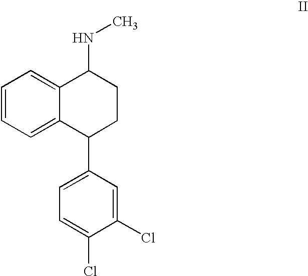
said process comprising:
(1) reacting 4-(3,4-dichlorophenyl)-3,4-dihydro-1-(2H)-naphthalenone of formula III: 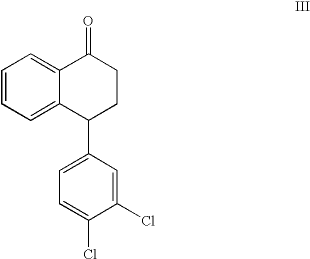
with monomethylamine in a solvent which is selected from the group consisting of amide solvents with a structure of general formula IV: 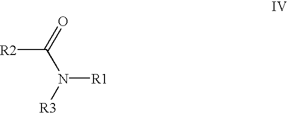
wherein R1 and R3 are independently hydrogen or C₁₋₆ alkyl, which can be substituted and R2 is hydrogen to obtain N-[4-(3,4-dichlorophenyl)-3,4-dihydro-1(2H)-naphthalenylidene]-methanamine of formula I: 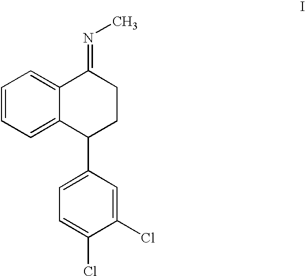
(2) hydrogenating said N-[4-(3,4-dichlorophenyl)-3,4-dihydro-1(2H)-naphthalenylidene]methanamine of formula I to obtain a mixture of racemic cis sertraline and racemic trans sertraline, and
(3) resolving said mixture of racemic cis sertraline and racemic trans sertraline to obtain said (1S-cis)-4-(3,4-dichlorophenyl)-1,2,3,4-tetrahydro-N-methyl-1-naphthalenamine or pharmaceutically acceptable salt thereof.

16. The process of claim 15, wherein said pharmaceutically acceptable salt is a hydrochloride.

17. The process of claim 15, wherein said mixture of racemic cis sertraline and racemic trans sertraline is resolved by forming a salt with mandelic acid.

18. The process of claim 17, wherein said pharmaceutically acceptable salt is a hydrochloride.

19. A pharmaceutical composition comprising (1S-cis)-4-(3,4-dichlorophenyl)-1,2,3,4-tetrahydro-N-methyl-1-naphthalenamine hydrochloride which is prepared by the process of claim 15.
